(19) United States
(12) Patent Application Publication
(10) Pub. No.: US 2010/0316713 A1
(43) Pub. Date: Dec. 16, 2010

(54) PHARMACEUTICAL COMPOSITION

(76) Inventors: Charu Kochhar, Basel (CH); Unmesh Deodhar, Binningen (CH); Aravind Kerudi, Bardez (IN); G.V. M. Babu, Bardez (IN)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/808,905

(22) PCT Filed: Dec. 19, 2008
(86) PCT No.: PCT/EP2008/068051

§ 371 Date: Aug. 25, 2010

(30) Foreign Application Priority Data
Dec. 21, 2007 (EP) 07150347.8

(51) Int. Cl. A61K 9/28; A61K 31/497

(52) U.S. Cl. 424/474; 514/252.17

(57) ABSTRACT

The application relates to solid pharmaceutical compositions suitable for oral administration comprising an indolylmaleimide derivative, process for their production and use of the pharmaceutical compositions.

The present invention relates to novel pharmaceutical compositions comprising 3-(1.H.-indol-3-yl)-4-[2-(4-methyl-piperazin-1-yl)-quinazolin-4-yl]-pyrrole-2,5-dione or a pharmaceutically acceptable salt thereof, process for their production and use of the pharmaceutical compositions.

3-(1.H.-indol-3-yl)-4-[2-(4-methyl-piperazin-1-yl)-quinazolin-4-yl]-pyrrole-2,5-dione is an indolylmaleimide derivative which has been shown to have an immunosuppressive activity.

3-(1.H.-indol-3-yl)-4-[2-(4-methyl-piperazin-1-yl)-quinazolin-4-yl]-pyrrole-2,5-dione is water sensitive and poorly water soluble. By water sensitive drug is meant an active agent which is highly soluble in water and in ethanol with a high powder-liquid ratio, e.g. a ratio of 10 mg/ml, and which may convert either to a free base hydrate, a solvate or an amorphous form in the presence of ethanol and/or water.

Pharmaceutical compositions comprising 3-(1.H.-indol-3-yl)-4-[2-(4-methyl-piperazin-1-yl)-quinazolin-4-yl]-pyrrole-2,5-dione are described in WO2006/092255, the content of which being incorporated herein by reference. In particular WO2006/092255 describes a tablet containing the acetate salt of 3-(1.H.-indol-3-yl)-4-[2-(4-methyl-piperazin-1-yl)-quinazolin-4-yl]-pyrrole-2,5-dione.

However there still exists a need for preparing an improved tablet containing 3-(1.H.-indol-3-yl)-4-[2-(4-methyl-piperazin-1-yl)-quinazolin-4-yl]-pyrrole-2,5-dione, or a pharmaceutically acceptable salt thereof.

In particular there is a need to prepare a tablet which is to be coated. Coating may be necessary for example to improve the aesthetic aspect of the tablet or permit to differentiate the available dosage strengths. Coating may also improve patient convenience and acceptance. To be coated, a tablet must have certain physical characteristics, such as hardness and low friability.

In accordance with the present invention it has now surprisingly been found that suitable tablets comprising 3-(1.H.-indol-3-yl)-4-[2-(4-methyl-piperazin-1-yl)-quinazolin-4-yl]-pyrrole-2,5-dione, or a pharmaceutically acceptable salt thereof, which are particularly stable, hard and convenient to administer are obtainable when the tablets comprise at least 2% by weight of lubricant, based on the total weight of the tablet core.

Usually the amount of lubricant contained in a tablet is comprised between 0.5 and 1.5% by weight, based on the total weight of the tablet core.

During tablet preparation, if the tablets are sticking to the tabletting tools (the punches), the tablets may then have an uneven surface, leading to coating problems, e.g. to an non homogeneous coating over the tablet surface. Furthermore such a sticking may lead to loss of potency or efficacy due to a loss of drug material. Unexpectedly it was found that this problem can be solved by using lubricant in a range higher than usually used in tablets, i.e. by using more than 0.5 to 2% by weight of lubricant.

Furthermore the present invention provides an improved tablet comprising 3-(1.H.-indol-3-yl)-4-[2-(4-methyl-piperazin-1-yl)-quinazolin-4-yl]-pyrrole-2,5-dione, or a pharmaceutically acceptable salt thereof, which is suitable to achieve high drug loads, and thus improve patient compliance. The tablet according to the present invention may show a high level of uniformity in the distribution of the drug as well as high stability.

According to the invention, there is provided a tablet, comprising from about 5 to about 90%, from about 10 to about 85%, from about 15 to about 80%, from about 20 to about 70%, from about 20 to about 55%, from about 25 to about 52%, from about 35 to about 52% by weight of 3-(1.H.-indol-3-yl)-4-[2-(4-methyl-piperazin-1-yl)-quinazolin-4-yl]-pyrrole-2,5-dione or a pharmaceutically acceptable salt thereof, based on the total tablet core weight.

One or more pharmaceutically acceptable carriers or diluents may be present in the tablet, e.g. at least one lubricant, at least one binder, at least one filler; and optionally at least one additional excipient selected from a disintegrant, a glidant; and a surfactant.

Lubricants according to the invention include e.g. Mg-, Al- or Ca-stearate, PEG 4000-8000, talc, sodium benzoate, glyceryl mono fatty acid, e.g. having a molecular weight of from 200 to 800 Daltons, e.g. glyceryl monostearate (e.g. Danisco, UK), glyceryl dibehenate (e.g. COMPRITOLAT™ 0888, Gattefossé France), glyceryl palmito-stearic ester (e.g. PRECIROL™, Gattefossé France), polyoxyethylene glycol (PEG, BASF), hydrogenated cotton seed oil (Lubitrab, Edward Mendell Co Inc), castor seed oil (Cutina HR, Henkel) and vinylpyrrolidone-vinyl acetate copolymer (e.g. Kollidon).

Preferably, magnesium stearate is used, either alone or in combination with another lubricant.

The composition of the invention contains from 2 to 15, preferably 2.5 to 12%, more preferably 3 to 10% by weight of a lubricant, based on the total weight of the composition, the total weight of the composition being the total tablet core weight.

The tablet of the invention comprises at least 2% by weight of lubricant, e.g. at least 2.5% by weight, e.g. at least 3% by weight, based on the total weight of the tablet core.

Thus, a particularly suitable tablet contains as lubricant from 2 to 15, e.g. 2.5. to 12, e.g. 3 to 10% by weight of magnesium stearate, based on the total weight of the tablet core.

In preferred compositions, lubricant is present in an amount of 3-5% by total weight of the tablet core, yet more preferably 3-4%.

Binders according to the invention include starches, e.g. potato, wheat or corn starch; hydroxypropyl cellulose; hydroxyethyl cellulose; hydroxypropylmethyl cellulose, e.g. hydroxypropylmethyl cellulose-Type 2910 USP, hypromellose, polyvinylpyrrolidone (e.g. POVIDONE K30), and Copovidone. Preferably, hydroxypropylmethyl cellulose, polyvinyl-pyrrolidone 30 or Copovidone, is used.

The composition of the invention may contain from 4 to 40% by weight, preferably from 4 to 35%, more preferably 5 to 30, even more preferably 5 to 20% by weight, of a binder based on the total weight of the tablet core. Thus, a particularly suitable tablet according to the invention contains as binder (a) from 5 to 20% by weight of copovidone.

Fillers according to the invention include e.g. lactose, especially lactose monohydrate, preferably lactose monohydrate (200 mesh) or lactose spray dried, microcrystalline cellulose, e.g. PH 102, PH 101, microcrystalline silicified cellulose, starch, calcium phosphate, or a saccharide, e.g. mannitol, maltodextrin or maltose, or a mixture thereof. Preferably, lactose spray dried, microcrystalline cellulose or microcrystalline silicified cellulose, more preferably lactose spray dried and microcrystalline cellulose or lactose spray dried and microcrystalline silicified cellulose is used.

The composition of the invention preferably contains from 15 to 65%, preferably 35 to 65% by weight of a filler, based on the total weight of the composition, the total weight of the tablet core. Thus, a particularly suitable solid pharmaceutical composition contains as filler (a) from 15 to 35, e.g. from 18 to 30% by weight of lactose, e.g. lactose spray dried, and from 10 to 35%, e.g. from 15 to 30% by weight of microcrystalline cellulose, based on the total weight of the tablet core.

Disintegrants according to the invention include e.g. natural starches, such as maize starch, potato starch, and the like; directly compressible starches, e.g. Sta-RX 1500; partially pregelatinized starch; modified starches, e.g. carboxymethyl starches and sodium starch glycolate; starch derivatives such as amylase, crosslinked polyvinylpyrrolidones, e.g. crospovidones, e.g. Polyplasdone® XL or Kollidon® CL, alginic acid or sodium alginate, methacrylic acid divinylbenzene copolymer salts, e.g. AMBERLITE i9 IRP-88, or cross-linked sodium carboxymethylcellulose, available as e.g. AC-DI-SOL; COMMAT; PRIMELLOSEF, PHARMACEL, EXPLOCEL, or NYMCEL ZSX. Preferably, a directly compressible starch, such as Sta-RX 1500, or a cross-linked polyvinylpyrrolidone, such as crospovidone, is used.

The composition of the invention preferably contains from 3 to 20% by weight, e.g. 5 to 15% by weight of a disintegrant, based on the total tablet core weight. Thus, a particularly suitable tablet contains as disintegrant from 3 to 20% by weight, e.g. 5 to 15% by weight of a directly compressible starch or a starch derivative, based on the total weight of core weight.

In another embodiment of the invention, the tablet may contain from 3 to 20%, e.g. from 3 to 10%, by weight of partially pregelatinized starch, based on the total weight of core weight.

The tablet of the invention may contain from 0 to 3% of a surfactant based on the total weight of the tablet core. Surfactants according to the invention include e.g. an anionic, cationic or non-ionic surfactant or a mixture thereof, e.g. sodium lauryl sulfate, cetrimide, a polysorbate or a sorbitan fatty acid ester, e.g. a sorbitan fatty acid ester from a fatty acid such as oleic, stearic or palmitin acid.

Glidants according to the invention include e.g. silica, colloidal silica, e.g. colloidal silicon dioxide, e.g. AEROSIL 200, magnesium trisilicate, powdered cellulose, starch and talc. Preferably, colloidal silicon dioxide is used.

The tablet of the invention preferably contains from 0.5 to 1% by weight of a glidant, based on the total weight of the tablet core. Thus, a particularly suitable tablet contains as glidant from 0.5 to 1% by weight of colloidal silicone dioxide, based on the total tablet core weight.

The tablet may optionally be coated, for instance with a coating comprising, a polysaccharide, e.g. cellulose, hydroxypropyl-methylcellulose, e.g. HMPC 603, polyoxyethylene glycol, e.g. PEG 6000 or PEG 8000, one or more dyers, carnauba wax, or an aluminium lake.

According to the invention, 3-(1.H.-indol-3-yl)-4-[2-(4-methyl-piperazin-1-yl)-quinazolin-4-yl]-pyrrole-2,5-dione is preferably in form of the acetate salt.

The tablet of the invention show good stability characteristics as indicated by standard stability trials, for example having a shelf life stability of up to one, two or three years, and even longer. Stability characteristics may be determined, e.g. by measuring decomposition products by HPLC analysis after storage for particular time periods, at various temperatures, e.g. 25°, 40° or 60° C.

The tablet of the present invention may be produced by standard processes, for instance by conventional mixing, compacting, granulating, compression, or coating with or without sugar. Procedures which may be used are known in the art, e.g. those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed. (Mack Publ., Co., 1970) or later editions.

In one aspect, the present invention relates to a process for producing the tablet of the invention, comprising: (a) mixing 3-(1.H.-indol-3-yl)-4-[2-(4-methyl-piperazin-1-yl)-quinazolin-4-yl]-pyrrole-2,5-dione or a pharmaceutically acceptable salt thereof, e.g. the acetate salt thereof, with a binder, a filler, a lubricant, and optionally a disintegrant and/or a glidant; (b) milling and/or granulating or compacting the mixture obtained in (a); and optionally (c) mixing the milled and/or granulated mixture obtained in (b) with a lubricant.

By using this process, a preparation having a good level of content and blend uniformity (i.e. a substantially uniform distribution of the drug throughout the composition), dissolution time and stability is obtained.

The tablet of the invention can be prepared by dry compression. This method involves the dry treatment of the drug substance, in which the drug is mixed with a part of the excipients, compressed either directly there or after screening and mixing with other suitable excipients. The process may be carried out by dry mixing the components. In this embodiment the milling step (b) may suitably comprise passing the mixture obtained in (a) through a screen, which preferably has a mesh size of 900 to 1000 μm.

The lubricant, e.g. magnesium stearate, is preferably pre-screened, e.g. with a 800 to 900 μm screen, before mixing.

Alternatively, a wet granulation, e.g. aqueous granulation process may be employed. Wet granulation has the advantage of providing an homogeneous material, i.e. granulates having a quite uniform particle size. Obtaining such an uniform material permits to obtain tablets with limited weight variation and uniform content. The compound 3-(1.H.-indol-3-yl)-4-[2-(4-methyl-piperazin-1-yl)-quinazolin-4-yl]-pyrrole-2,5-dione show high degradation in water, which renders aqueous granulation difficult. Surprisingly, it has been found that the problem can be overcome with a new method of manufacture in which the contact of the drug substance with water is prevented. The filler, binder, and optionally the disintegrant, are granulated together using an aqueous solution, e.g. an aqueous solution of the binder, in a high shear mixture. The granulates thus obtained are dried, milled and then mixed with a lubricant. 3-(1.H.-indol-3-yl)-4-[2-(4-methyl-piperazin-1-yl)-quinazolin-4-yl]-pyrrole-2,5-dione, optionally mixed to at least one further excipient selected from a filler, a disintegrant, a glidant, and a binder, is mixed to the granulates. A lubricant may then be added. Then the granulates are compressed into tablets.

The tablet of the invention may be used for the treatment or prevention of the diseases for which the active agent it contains, is useful. Thus, the tablet of the invention may be used in the treatment and/or prevention of diseases or disorders mediated by T lymphocytes and/or PKC, e.g. acute or chronic rejection of organ or tissue allo- or xenografts, atherosclerosis, vascular occlusion due to vacular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock. 3-(1.H.-indol-3-yl)-4-[2-(4-methyl-piperazin-1-yl)-quinazolin-4-yl]-pyrrole-2,5-dione is also useful in the treatment and/or prevention of T-cell mediated acute or chronic inflammatory diseases or disorders or autoimmune diseases, e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

For the above uses the required dosage will of course vary depending on the particular condition to be treated and the effect desired. In general, satisfactory results are indicated to be obtained systemically at daily dosages of from about 0.1 to about 100 mg/kg body weight. An indicated daily dosage in the larger mammal, e.g. humans, is in the range from about 0.5 mg to about 2000 mg, conveniently administered, for example, in divided doses up to four times a day.

The tablet of the invention may be administered in conjunction with a co-agent depending on the diseases or disorders to be treated. For example it may be administered in conjunction, either simultaneously or in sequence, with other drugs in immunomodulating regimens or other anti-inflammatory agents e.g. for the treatment or prevention of allo- or xenograft acute or chronic rejection or inflammatory or autoimmune disorders. For example, it may be used in combination with cyclosporines, or ascomycines or their immunosuppressive analogs or derivatives, e.g. cyclosporin A, cyclosporin G, FK-506, ABT-281, ASM 981; an mTOR inhibitor, e.g. rapamycin, 40-O-(2-hydroxy-ethyl)-rapamycin, CCI779, ABT578, biolimus-7, biolimus-9, TAFA-93, AP23573, AP23464 or AP23841 etc.; corticosteroids; cyclophosphamide; azathioprene; methotrexate; a S1P receptor modulator, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CD3, CD4, CD 11a/CD18, CD7, CD25, CD 27, B7, CD40, CD45, CD58, CD 137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists. The tablet of the invention may also be administered in conjunction with, e.g. simultaneously or in sequence, an antiproliferative drug, e.g. a chemotherapeutic drug, e.g. in cancer treatment, or with an anti-diabetic drug in diabetes therapy. Dosages of the co-administered immunosuppressant, immunomodulatory, anti-inflammatory, antiproliferative or anti-diabetic agent may vary depending on the type of the co-agent used, on the specific drug employed, on the condition being treated and so forth.

In accordance with the foregoing the present invention further provides:
1. A tablet containing 3-(1.H.-indol-3-yl)-4-[2-(4-methyl-piperazin-1-yl)-quinazolin-4-yl]-pyrrole-2,5-dione or a pharmaceutically acceptable salt thereof, as defined above, for use in preventing or treating disorders or diseases mediated by T lymphocytes and/or PKC, e.g. such as indicated above, in a subject in need of such treatment.
2.1 A method for preventing or treating disorders or diseases mediated by T lymphocytes and/or PKC, e.g. such as indicated above, in a subject in need of such treatment, which method comprises administering to said subject an effective amount of a tablet as defined above.
2.2 A method for preventing or treating acute or chronic transplant rejection or T-cell mediated inflammatory or autoimmune diseases, e.g. as indicated above, in a subject in need of such treatment, which method comprises administering to said subject an effective amount of a tablet as defined above.
2.3 A method for preventing or treating disorders or diseases mediated by T lymphocytes and/or PKC, e.g. such as indicated above, in a subject in need of such treatment, which method comprises co-administration, e.g. concomitantly or in sequence, of a therapeutically effective amount of a tablet containing 3-(1.H.-indol-3-yl)-4-[2-(4-methyl-piperazin-1-yl)-quinazolin-4-yl]-pyrrole-2,5-dione, as defined above, and a second drug substance, said second drug substance being an immunosuppressant, immunomodulatory, anti-inflammatory, antiproliferative or anti-diabetic drug, e.g. as indicated above.
3. A therapeutic combination, e.g. a kit, comprising a) a tablet containing 3-(1.H.-indol-3-yl)-4-[2-(4-methyl-piperazin-1-yl)-quinazolin-4-yl]-pyrrole-2,5-dione, as defined above, and b) at least one second agent selected from an immunosuppressant, immunomodulatory, anti-inflammatory, antiproliferative and anti-diabetic drug. Component a) and component b) may be used concomitantly or in sequence. The kit may comprise instructions for its administration.
4. Use of a tablet containing 3-(1.H.-indol-3-yl)-4-[2-(4-methyl-piperazin-1-yl)-quinazolin-4-yl]-pyrrole-2,5-dione, as defined above, for the preparation of a medicament for the prevention or treatment of disorders or diseases mediated by T lymphocytes and/or PKC, e.g. such as indicated above.

The invention will now be described with reference to the following specific embodiments.

### EXAMPLE 1

### Dry Compression Followed by Coating

The drug substance is mixed with the diluent, binder, disintegrant, glidant (positions 1-6) and mixed in a free fall mixer. This premix is mixed again with the lubricant (Pos 7) and compressed directly into tablets of different strengths.

TABLE 1

| S. | | Amount |
|---|---|---|
| No. | Ingredients | (in %)* |
| 1 | 3-(1.H.-indol-3-yl)-4-[2-(4-methyl- | 38% |
| | piperazin-1-yl)-quinazolin-4-yl]- | |
| | pyrrole-2,5-dione acetate | |
| 2 | Microcrystalline cellulose | 20% |
| 3 | Lactose | 26% |
| 4 | Copovidone | 6% |
| 5 | Crospovidone | 5% |
| 6 | Colloidal silicon dioxide | 1% |
| 7 | Magnesium stearate | 4% |
| | Core tablet weight | |
| 8 | Hypromellose based coating | 4% |

Film coating

| | | |
|---|---|---|
| 9 | HPMC | 4% of tablet core |

*amount based on the tablet core weight

### EXAMPLE 1a

### Dry Compression Followed by Coating

The drug substance is mixed with the diluent, binder, disintegrant, glidant (positions 1-6) and mixed in a free fall mixer. This premix is mixed again with the lubricant (Pos 7) and compressed directly into tablets of different strengths. Examples of three compositions obtainable via this method are shown in Table 1a.

TABLE 1a

| S. | | Amount (in %)* | | |
|---|---|---|---|---|
| No. | Ingredients | 1a.1 | 1a.2 | 1a.3 |
| 1 | 3-(1.H.-indol-3-yl)-4-[2-(4-methyl- | 38% | 38% | 38% |
| | piperazin-1-yl)-quinazolin-4-yl]- | | | |
| | pyrrole-2,5-dione acetate | | | |
| 2 | Microcrystalline cellulose | 15% | 25% | 15% |
| 3 | Lactose | 28% | 16% | 34% |
| 4 | Copovidone | 9% | 9% | 3% |
| 5 | Crospovidone | 6% | 6% | 6% |
| 6 | Colloidal silicon dioxide | 1% | 1% | 1% |
| 7 | Magnesium stearate | 3% | 5% | 3% |
| | Core tablet weight | | | |

*amount based on the tablet core weight

These tablets are then coated with a non-functional coating agent.

### EXAMPLE 2

### Dry Compression Followed by Coating

The drug substance is mixed with the diluent, binder, disintegrant, glidant (positions 1-5), mixed together with the lubricant (Position 6), is compressed at first stage into a tablet or into a ribbon, which is screened through a suitable sieve to give a granulation. This granulation is mixed again with another part of diluent, binder, disintegrant and glidant (Pos 7-10) and compressed into tablets after adding a suitable lubricant to the mixture.

TABLE 2

| S. | | |
|---|---|---|
| No. | Ingredients | Amount (in %)* |
| 1 | 3-(1.H.-indol-3-yl)-4-[2-(4-methyl- | 38% |
| | piperazin-1-yl)-quinazolin-4-yl]- | |
| | pyrrole-2,5-dione acetate | |
| 2 | Microcrystalline cellulose | 20% |
| 3 | Lactose | 25% |
| 4 | Crospovidone | 2% |
| 5 | Colloidal silicon dioxide | 1% |
| 6 | Magnesium stearate | 2% |
| 7 | Microcrystalline cellulose | 5% |
| 8 | Crospovidone | 3% |
| 9 | Colloidal silicon dioxide | 1% |
| 10 | Partially Pregelatinised Starch | 3% |
| 11 | Magnesium stearate | 3% |

Film coating

| | | |
|---|---|---|
| 12 | HPMC based premix | 4% of tablet core |

*amount based on the tablet core weight

### EXAMPLE 3

### Wet Granulation

The excipients (positions 1, 2 and 4) are granulated together using a solution of the binder in purified water (position 3) in a high shear mixture. The granulate is dried, screened and mixed with lubricant (position 5). To this, the drug substance (Pos 6) along with excipients (7, 8, 9, 10) are added, mixed together, mixed again with the rest of the lubricant (11) and compressed into tablets. These tablets are then coated with a non-functional coating agent.

TABLE 3

| | | |
|---|---|---|
| Pos. | Ingredients | Amount (in %)* |

Internal phase

| | | |
|---|---|---|
| 1 | Lactose monohydrate | 31% |
| 2 | Maize Starch | 3% |
| 3 | Povidone K-30 | 4% |
| 4 | Crospovidone | 2% |
| 5 | Magnesium stearate | 2% |
| | Purified water | q.s. |

External Phase

| | | |
|---|---|---|
| 6 | 3-(1.H.-indol-3-yl)-4-[2-(4-methyl- | 38% |
| | piperazin-1-yl)-quinazolin-4-yl]- | |
| | pyrrole-2,5-dione acetate | |
| 7 | Microcrystalline cellulose | 5% |
| 8 | Crospovidone | 7% |
| 9 | Colloidal silicon dioxide | 1% |
| 10 | Kollidon VA-64 | 6% |
| 11 | Magnesium stearate | 2% |

Core tablet

| | | |
|---|---|---|
| 12 | HPMC | 4% of tablet core |

*amount based on the tablet core weight

1. A solid pharmaceutical composition suitable for oral administration comprising 3-(1.H.-indol-3-yl)-4-[2-(4-methyl-piperazin-1-yl)-quinazolin-4-yl]-pyrrole-2,5-dione or a pharmaceutically acceptable salt thereof and at least one lubricant, wherein the composition comprises from 2.5 to 12% by weight of lubricant, and the composition is in form of a tablet, the total weight of the composition being the total tablet core weight.

2. A composition according to claim 1 wherein the composition comprises from 3 to 10% by weight of lubricant, based on the total tablet core weight.

3. A composition according to claim 1 wherein the composition comprises 5 to 90% by weight of the compound, based on the total tablet core weight.

4. A composition according to claim 1 further comprising at least one binder.

5. A composition according to claim 4 wherein the composition comprises from 4 to 40% by weight of the binder, based on the total tablet core weight.

6. A composition according to claim 1 further comprising at least one filler.

7. A composition according to claim 1 comprising at least one further excipient selected from a disintegrant, a glidant and a surfactant.

8. A composition according to claim 1, which comprises the acetate salt of 3-(1.H.-indol-3-yl)-4-[2-(4-methyl-piperazin-1-yl)-quinazolin-4-yl]-pyrrole-2,5-dione.

9. A composition according to claim 1 for use in preventing or treating disorders or diseases mediated by T lymphocytes and/or PKC in a subject in need of such treatment.

10. A process for producing a tablet according to claim 1 comprising: (a) mixing 3-(1.H.-indol-3-yl)-4-[2-(4-methyl-piperazin-1-yl)-quinazolin-4-yl]-pyrrole-2,5-dione, or a pharmaceutically acceptable salt thereof, with a binder, a filler, and optionally, at least one further excipient selected from a disintegrant, and a glidant; (b) mixing, compacting, milling, granulating, drying or compacting the mixture obtained in (a); (c) mixing the mixture obtained in (b) with a lubricant; (d) tableting and (e) optionally coating.

11. A process for producing a tablet according to claim 1 comprising:
(a) mixing a filler, a binder, and optionally a disintegrant, in an aqueous solution and granulating the mixture obtained;
(b) drying and milling the granulates obtained in (a) and mixing them with a lubricant;
(c) optionally mixing 3-(1.H.-indol-3-yl)-4-[2-(4-methyl-piperazin-1-yl)-quinazolin-4-yl]-pyrrole-2,5-dione, or a pharmaceutically acceptable salt thereof, to at least one further excipient selected from a filler, a disintegrant, a glidant, a binder and a lubricant
(d) mixing either directly 3-(1.H.-indol-3-yl)-4-[2-(4-methyl-piperazin-1-yl)-quinazolin-4-yl]-pyrrole-2,5-dione, or a pharmaceutically acceptable salt thereof, or the mixture obtained under step (c) to the granulates obtained in step (b), optionally in presence of a lubricant;
(e) tableting the mixture obtained in step (b), (c) or (d) and
(f) optionally coating.

12. A solid pharmaceutical composition suitable for oral administration comprising the acetate sale of 3-(1.H.-indol-3-yl)-4-[2-(4-methyl-piperazin-1-yl)-quinazolin-4-yl]-pyrrole-2,5-dione and at least one lubricant wherein the composition comprises from 3 to 5% by weight of lubricant, and the composition is in the form of a tablet, the total weight of the composition being the total tablet core weight.
